(19) United States
(12) Patent Application Publication
(10) Pub. No.: US 2016/0106888 A1
(43) Pub. Date: Apr. 21, 2016

(54) Collagen Material and Composites for Ocular Application

(71) Applicant: The Johns Hopkins University, Baltimore, MD (US)

(72) Inventors: Xiomara Calderon-Colon, Laurel, MD (US); Annie M. Dunn, Ellicott City, MD (US); Marcia W. Patchan, Columbia, MD (US); Morgana M. Trexler, Baltimore, MD (US)

(21) Appl. No.: 14/918,856

(22) Filed: Oct. 21, 2015

Related U.S. Application Data
(60) Provisional application No. 62/066,435, filed on Oct. 21, 2014.

(51) Int. Cl. A61L 27/48; A61F 2/02; D01D 5/00; A61L 27/54; A61L 27/24

(52) U.S. Cl. CPC A61L 27/48; A61L 27/54; A61L 27/24; D01D 5/003; A61F 2/02; A61L 2430/16; D10B 2211/06; D10B 2509/00

(57) ABSTRACT

A method for preparing a collagen membrane includes applying an influence of an electric field to a collagen solution positioned between capacitor plates; adding a buffer solution to the acidic collagen solution to form a collagen gel; assembling a plurality of collagen gel layers; and performing a dehydrothermal cross-link on the plurality of collagen gel layers to form a cross-linked collagen membrane.

### CROSS-REFERENCE TO RELATED APPLICATIONS

This application claims the benefit of U.S. Provisional Application No. 62/066,435 filed on Oct. 21, 2014, the entire contents of which are hereby incorporated herein by reference.

### TECHNICAL FIELD

Example embodiments relate generally to methods for preparing collagen membranes.

### BACKGROUND

The cornea is disposed at the outermost part of the optical system constituting the eyeball and is a clear tissue that does not contain blood vessels. The cornea and tear fluid form a smooth eyeball surface, thereby obtaining satisfactory eyesight. Keratoconjunctival epithelial cells are constantly in contact with the external world and have a function of protecting the eyeball from foreign matters such as microorganisms in the external world and rays of light such as ultraviolet rays. That is, the keratoconjunctival epithelial cells play an extremely important role in order to maintain the clarity of the cornea and to protect the whole eyeball to maintain homeostasis. When the cornea becomes cloudy and loses its clarity because of a disease such as keratitis, corneal ulcer, or perforation of the cornea, eyesight is permanently degraded. As a therapy for the degradation of eyesight caused by such a clouding of the cornea, corneal transplantation is performed. In the corneal transplantation, a cornea of a patient from which the clarity is lost is removed, and a new clear cornea is transplanted. By performing such transplantation, clarity is recovered, and eyesight can be restored again.

Because native cornea is made up of aligned collagen fibers, collagen is an ideal material for ocular reconstruction. However, existing applications of collagen for ocular reconstruction have been limited by poor optical and mechanical properties. Specifically, conventional collagen materials used for ocular reconstruction do not provide sufficient visual clarity or ease of handling and suturing to the eye.

Therefore there remains a need in the art for a method for preparing a collagen membrane that produces a transparent collagen material that provides ease of handling for ocular applications.

### BRIEF SUMMARY

One or more example embodiments may address one or more of the aforementioned problems. Certain example embodiments provide a method for preparing a collagen membrane for optical applications. In accordance with certain embodiments, the method may comprise applying an influence of an electric field to an acidic collagen solution positioned between two capacitor plates, adding a buffer solution to the acidic collagen solution to form a collagen gel, assembling a plurality of collagen gel layers, and performing a dehydrothermal cross-link on the plurality of collagen gel layers to form a cross-linked collagen membrane.

In another aspect, a method for preparing a collagen composite is provided. In accordance with certain embodiments, the method may comprise forming a collagen solution, coaxially electrospinning the collagen solution and at least one polymer to form collagen composite nanofibers, and collecting the collagen composite nanofibers.

In yet another aspect, a collagen composite is provided. In accordance with certain embodiments, the collagen composite may comprise a collagen shell and a polymer core. The polymer core may comprise at least one of a therapeutic polymeric composition, a sensory polymeric composition, or any combination thereof. Moreover, the collagen shell and the polymer core may be coaxially electrospun together to form the collagen composite.

### BRIEF DESCRIPTION OF THE DRAWING(S)

Having thus described example embodiments in general terms, reference will now be made to the accompanying drawings, which are not necessarily drawn to scale, and wherein:

FIG. 1 illustrates a block diagram of a method for preparing a collagen membrane including the optional steps of performing a chemical cross-linking reaction on the cross-linked collagen membrane to form a double cross-linked collagen membrane and rehydrating the double cross-linked collagen membrane according to an example embodiment.

FIGS. 2A-2C illustrate SEM images of collagen membranes according to an example embodiment.

FIG. 3 illustrates a TEM image of a collagen membrane cross section according to an example embodiment.

FIG. 4 illustrates the optical properties of collagen membranes according to an example embodiment.

FIG. 5 illustrates a block diagram of a method for preparing a collagen composite including the optional steps of hydrating the collagen composite nanofibers and performing a cross-link reaction on the collagen composite nanofibers according to an example embodiment.

FIG. 6 illustrates an electrospinning set-up according to an example embodiment.

FIGS. 7A and 7B illustrate SEM images of electrospun collagen nanofibers according to an example embodiment.

FIGS. 8A and 8B illustrate the structure and formation of a collagen composite according to an example embodiment.

### DETAILED DESCRIPTION

Some example embodiments now will be described more fully hereinafter with reference to the accompanying drawings, in which some, but not all embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout. As used in the specification, and in the appended claims, the singular forms “a”, “an”, “the”, include plural referents unless the context clearly dictates otherwise.

I. Method for Preparing a Collagen Membrane

In some example embodiments, a method for preparing a collagen membrane is provided. In general, the method for preparing the collagen membrane, according to certain embodiments, may include applying the influence of an electric field to an acidic collagen solution positioned between two capacitor plates, adding a buffer solution to the acidic collagen solution to form a collagen gel, assembling a plurality of collagen gel layers, performing a dehydrothermal cross-link on the plurality of collagen gel layers to form a cross-linked collagen membrane, and rehydrating the cross-linked collagen membrane. In further embodiments, for instance, the method may further comprise performing a chemical cross-linking reaction on the cross-linked collagen membrane to form a double cross-linked collagen membrane and rehydrating the double cross-linked collagen membrane.

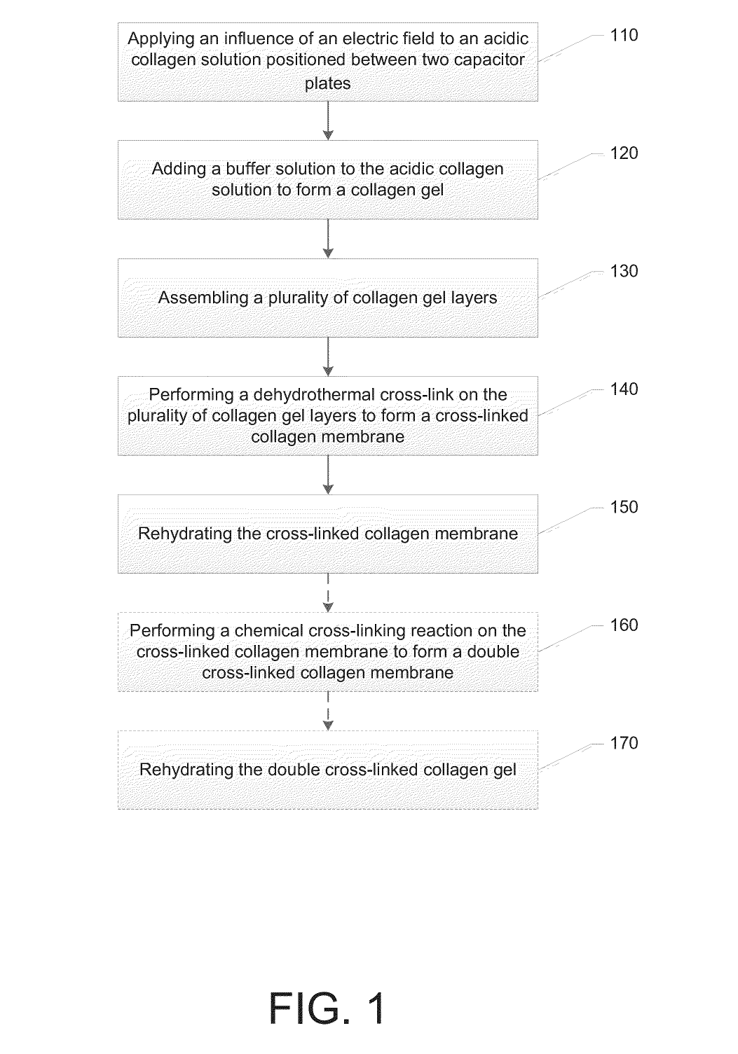

FIG. 1, for example, illustrates a block diagram of a method for preparing a collagen membrane including the optional steps performing a chemical cross-linking reaction on the cross-linked collagen membrane to form a double cross-linked collagen membrane and rehydrating the double cross-linked collagen membrane according to an example embodiment. As shown in FIG. 1, the exemplary method includes applying an influence of an electric field to an acidic collagen solution positioned between two capacitor plates at operation 110, adding a buffer solution to the acidic collagen solution to form a collagen gel at operation 120, assembling a plurality of collagen gel layers at operation 130, performing a dehydrothermal cross-link on the plurality of collagen gel layers to form a cross-linked collagen membrane 140, and rehydrating the cross-linked collagen membrane at operation 150. The exemplary method may also include the optional steps of performing a chemical cross-linking reaction on the cross-linked collagen membrane to form a double cross-linked collagen membrane at operation 160 and rehydrating the double cross-linked collagen membrane at operation 170.

In accordance with certain embodiments, the capacitor may include an upper plate and a lower plate (i.e. the capacitor plate on which the collagen solution is positioned). In some example embodiments, both the upper plate and the lower plate may be flat, horizontal plates situated substantially parallel to each other. The electric field may be formed by transmitting energy from the upper plate to the lower plate. When energy is transmitted from the upper plate through the acidic collagen solution to the lower plate, some energy may have an influence on the acidic collagen solution, and some energy may be received by the lower plate. As the collagen solution is influenced by the energy, a buffer may cause a change in the collagen solution pH from an acidic pH to a neutral pH. In this regard, by controlling the pH of the collagen solution, the charges along the collagen molecules may be controlled. During the gelation, collagen molecules may begin to assemble into a network structure that leads to the formation of a gel-like material. In some embodiments, for instance, a plurality of collagen gels may be assembled together into a plurality of collagen gel layers. The plurality of collagen gel layers may then be dehydrothermally cross-linked to form a collagen membrane that comprises a plurality of collagen gel layers.

According to certain embodiments, for instance, applying the influence of the electric field to the acidic collagen solution positioned between the capacitor plates may comprise applying the influence of the electric field to a dialyzed acid collagen solution positioned between the capacitor plates. Moreover, in certain embodiments, for instance, the capacitor plate may comprise a stainless steel plate. In further embodiments, for example, the collagen solution may be poured onto a metal mold inside a Petri dish, covered, and placed between the two plates.

In accordance with an example embodiment, for example, applying the influence of the electric field to the collagen solution positioned between the capacitor plates may comprise applying a voltage between parallel capacitor plates for a time period of about 1 hour to about 15 hours. In other embodiments, for instance, applying the influence of the electric field to the collagen solution positioned between the capacitor plates may comprise applying a voltage between parallel capacitor plates for a time period of about 5 hours to about 12 hours. In further embodiments, for example, applying the influence of the electric field to the collagen solution positioned between the capacitor plates may comprise applying a voltage between parallel capacitor plates for a time period of about 6 hours to about 10 hours. In certain embodiments, for instance, applying the influence of the electric field to the collagen solution positioned between the capacitor plates may comprise applying a voltage between parallel capacitor plates for a time period of about 8 hours. As such, in certain embodiments, applying the influence of the electric field to the collagen solution positioned between the capacitor plates may comprise applying a voltage between parallel capacitor plates for a time period from at least about any of the following: 1, 2, 3, 4, 5, 6, 7, and 8 hours and/or at most about 15, 14, 13, 12, 11, 10, 9 and 8 hours (e.g., about 3-12 hours, about 7-9 hours, etc.).

According to certain embodiments, for example, applying the influence of the electric field to the collagen solution positioned between the capacitor plates may comprise applying a voltage at an electrode distance from about 1 cm and about 5 cm. In other embodiments, for instance, applying the influence of the electric field to the collagen solution positioned between the capacitor plates may comprise applying a voltage at an electrode distance from about 2 cm and about 4 cm. In further embodiments, for example, applying the influence of the electric field to the collagen solution positioned between the capacitor plates may comprise applying a voltage at an electrode distance from about 2 cm and about 3 cm. In certain embodiments, for instance, applying the influence of the electric field to the collagen solution positioned between the capacitor plates may comprise applying a voltage at an electrode distance of about 1 cm. As such, in certain embodiments, applying the influence of the electric field to the collagen solution positioned between the capacitor plates may comprise applying a voltage at an electrode distance from at least about any of the following: 1, 2, 3, 4, and 5 cm and/or at most about 5, 4.5, 4, 3.5, 3, 2.5, 2, 1.5, and 1 cm (e.g., about 1-3 cm, about 1-4 cm, etc.).

In certain embodiments, for example, applying the influence of the electric field to the collagen solution positioned between the capacitor plates may comprise applying a voltage from about 1 V to about 15 V. In other embodiments, for instance, applying the influence of the electric field to the collagen solution positioned between the capacitor plates may comprise applying a voltage from about 4 V to about 12 V. In further embodiments, for example, applying the influence of the electric field to the collagen solution positioned between the capacitor plates may comprise applying a voltage from about 5 V to about 10 V. In some embodiments, for instance, applying the influence of the electric field to the collagen solution positioned between the capacitor plates may comprise applying a voltage of about 5 V. In other embodiments, for example, applying the influence of the electric field to the collagen solution positioned between the capacitor plates may comprise applying a voltage of about 10 V. As such, in certain embodiments, applying the influence of the electric field to the collagen solution positioned between the capacitor plates may comprise applying a voltage from at least about any of the following: 1, 2, 3, 4, 5, 6, 7, 8, 9, and 10 V and/or at most about 15, 14, 13, 12, 11, 10, 9, 8, 7, 6, and 5 V (e.g., about 5-12 V, about 9-10 V, etc.). However, the electrode distance and the applied voltage may vary depending on the desired electric field. For example, an electrode distance of 1 cm and an applied field of either 5 V or 10 V may produce an electric field of 5 V/cm and 10 V/cm respectively.

Moreover, in some embodiments, for instance, the collagen solution may be exposed to the influence of the electric field for a given period of time at a temperature from about 15° C. to about 45° C. In other embodiments, for example, the collagen solution may be exposed to the influence of the electric field for a given period of time at a temperature from about 20° C. to about 40° C. In certain embodiments, for instance, the collagen solution may be exposed to the influence of the electric field for a given period of time at a temperature of about 23° C. In further embodiments, for example, the collagen solution may be exposed to the influence of the electric field for a given period of time at a temperature of about 37° C. As such, in certain embodiments, the collagen solution may be exposed to the influence of the electric field for a given period of time at a temperature from at least about any of the following: 15, 18, 20, 21, 22, 23, 25, 30, 35, and 37° C. and/or at most about 45, 43, 40, 39, 38, 37, 35, 30, 25, and 23° C. (e.g., about 23-37° C., about 21-35° C., etc.).

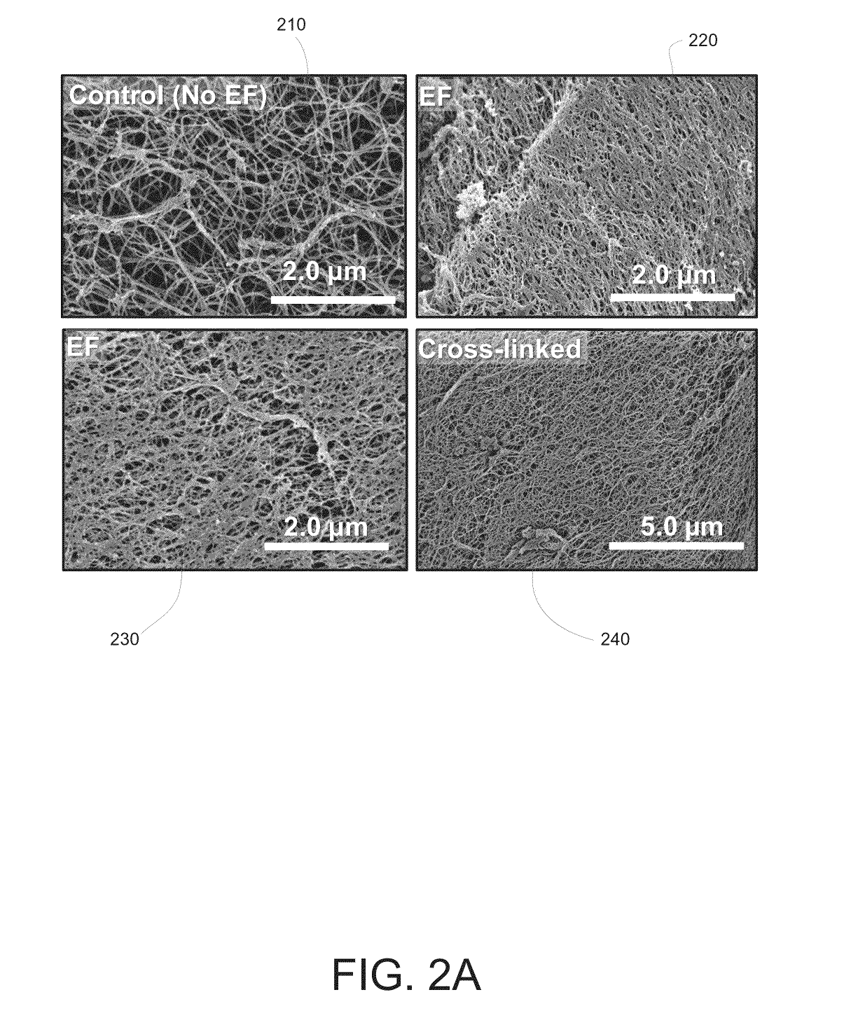

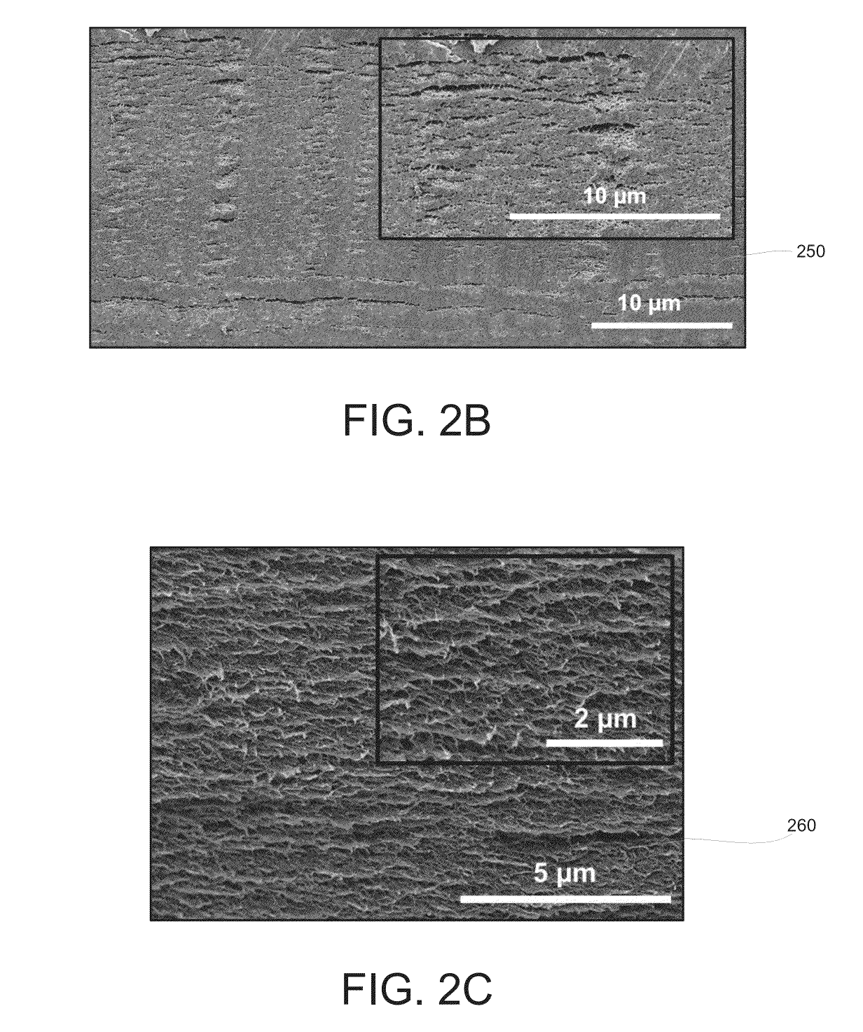

In this regard, applying the influence of the electric field to the collagen solution may result in different arrangement of collagen fibrils in the collagen membrane than those collagen fibrils that appear in conventional collagen materials. For example, FIGS. 2A-2C illustrate SEM images of collagen membranes according to an example embodiment. As shown in FIG. 2A, which illustrates a top view of the collagen membrane, the influence of the electric field on the collagen solution for 8 hours resulted in a different arrangement of collagen fibrils 220, 230 than the control 210 that was not exposed to the influence of the electric field. Moreover, dehydrothermal cross-linking of the collagen membrane 240 does not alter the collagen membrane morphology. Cross sections of cross-linked collagen membranes are also visible at two different magnifications in FIGS. 2B and 2C.

According to certain embodiments, for instance, applying the influence of the electric field to the collagen solution may result in collagen nanofibers comprising a fibril diameter from about 20 nm to about 50 nm. In other embodiments, for instance, the collagen nanofibers may comprise a fibril diameter from about 22 nm to about 45 nm. In further embodiments, for example, the collagen nanofibers may comprise a fibril diameter from about 24 nm to about 44 nm. In some embodiments, for instance, the collagen nanofibers may comprise a fibril diameter from about 25 nm to about 42 nm. As such, in certain embodiments, the collagen nanofibers may comprise a fibril diameter from at least about any of the following: 20, 21, 22, 23, 24, and 25 nm and/or at most about 50, 49, 48, 47, 46, 45, 44, 43, and 42 nm (e.g., about 20-42 nm, about 25-50 nm, etc.).

In accordance with certain embodiments, for instance, dehydrothermal cross-linking and rehydrating the membrane at least once may improve the membrane's mechanical properties (e.g., by controlling the humidity, temperature, and time). In some embodiments, for example, the membrane may be chemically cross-linked and rehydrated at least twice. According to certain embodiments, for instance, the collagen cross-linkers may comprise at least one of 1-ethyl-3-(3 dimethyl-aminopropyl)-1-carbodiimide hydrochloride (EDC), N-hydroxysuccinimide (NHS), riboflavin, 2-Hydroxy-4′-(2-hydroxyethoxy)-2-methylpropiophenone, or any combination thereof.

According to certain embodiments, for instance, the collagen membrane may comprise a thickness from about 100 microns to about 600 microns. In other embodiments, for example, the collagen membrane may comprise a thickness from about 150 microns to about 500 microns. In further embodiments, for instance, the collagen membrane may comprise a thickness from about 250 microns to about 500 microns. In some embodiments, for example, the collagen membrane may comprise a thickness from about 300 microns to about 500 microns. In certain embodiments, for instance, the collagen membrane may comprise a thickness of about 500 microns. As such, in certain embodiments, the collagen membrane may comprise a thickness from at least about any of the following: 100, 150, 200, 250, 300, 350, 400, 450, and 500 microns and/or at most about 600, 590, 580, 570, 560, 550, 540, 530, 520, 510, and 500 microns (e.g., about 250-500 microns, about 300-550 microns, etc.).

Accordingly, in certain embodiments, for example, the thickness may be substantially the same as a corneal thickness (i.e. 500 microns). As such, in some embodiments, for instance, the collagen membrane may be configured to be sutured to an eye (e.g., by being shaped as a disk via, for example, a biopsy punch and/or a mold having the curvature of the eye). In this regard, the collagen membrane may be of an appropriate thickness for use in a full human corneal replacement (i.e. about 500 microns), a partial human corneal replacement (i.e. about 250 microns), a full animal (e.g., rabbit) corneal replacement (i.e. about 300 microns), a partial animal (e.g., rabbit) corneal replacement (i.e. about 150 microns) and/or the like.

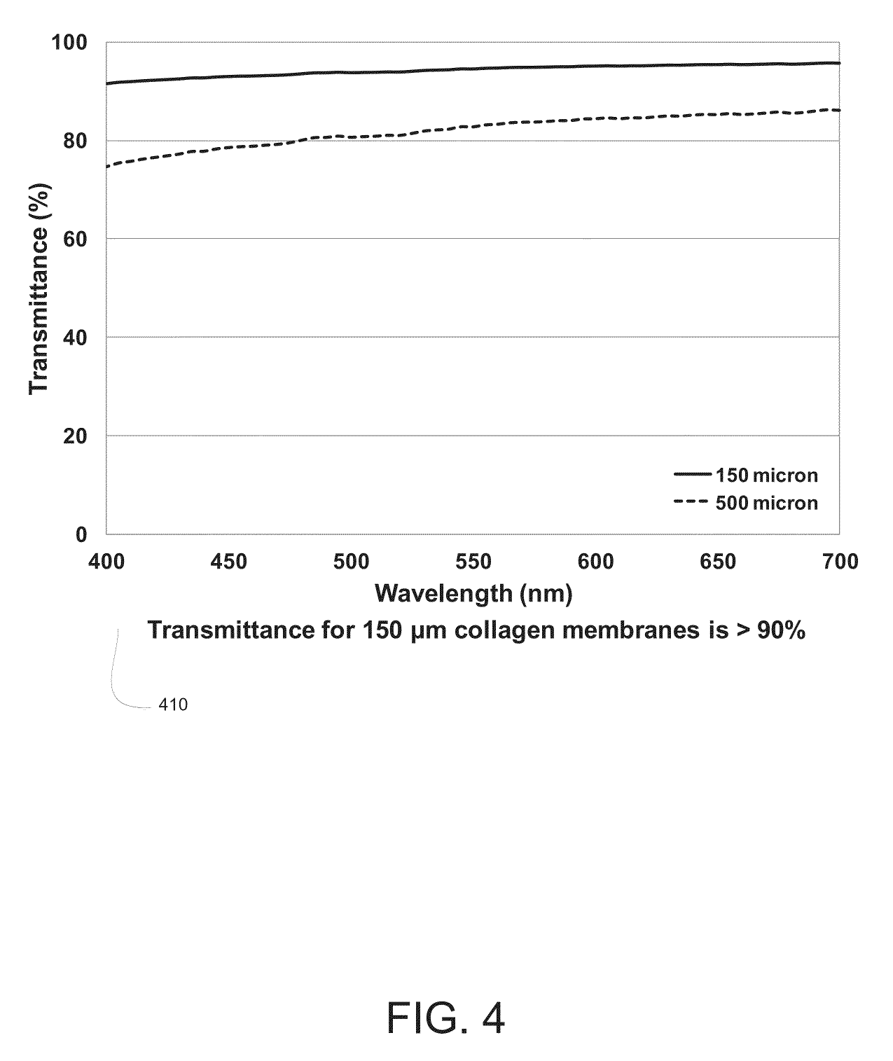

In accordance with an example embodiment, for instance, the collagen membrane may be substantially transparent. For example, FIG. 4 illustrates the optical properties of collagen membranes according to an example embodiment. As shown in FIG. 4, transmittance at 550 nm for a 150 micron collagen membrane is greater than 90%. As such, the collagen membrane according to an example embodiment is substantially transparent.

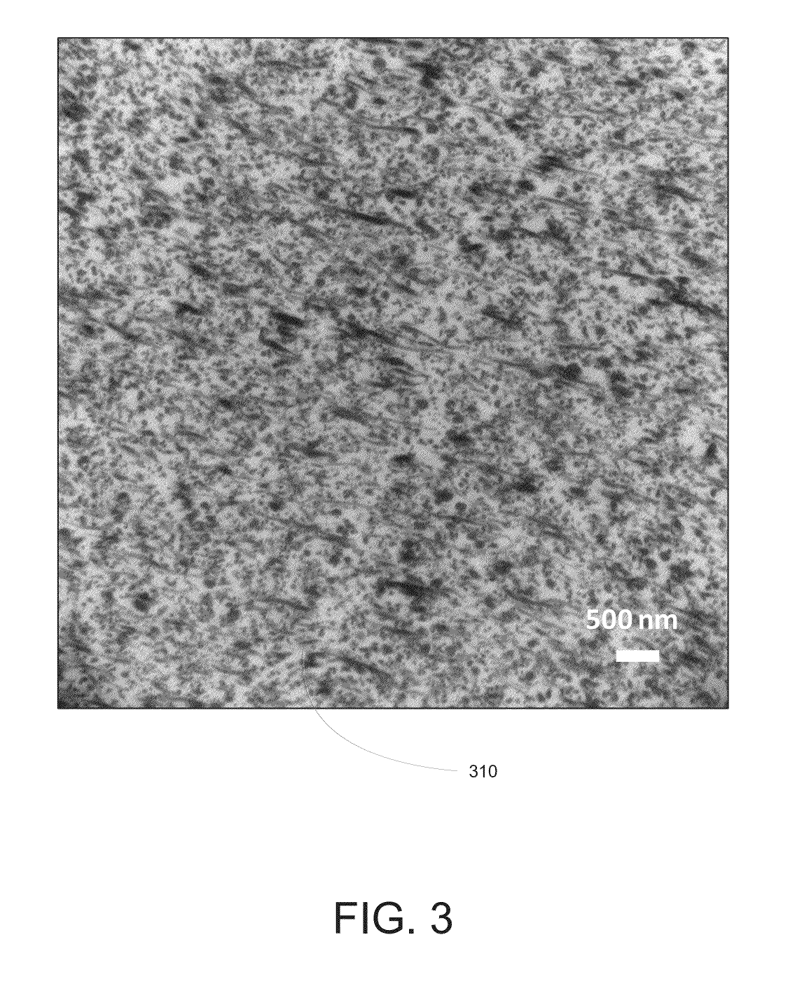

According to certain embodiments, for example, the collagen membrane may comprise a plurality of collagen fibrils, and the plurality of collagen fibrils may be substantially aligned. For example, FIG. 3 illustrates a collagen membrane cross section via a TEM image 310 according to an example embodiment. As shown in FIG. 3, those collagen membranes formed under the influence of an electric field exhibit a distinct collagen fibril arrangement.

II. Method for Preparing Electrospun Collagen Composite Nanofibers

In another aspect, a method for preparing a collagen composite is provided. In general, the method for preparing a collagen composite, according to certain embodiments, may include forming a collagen solution, coaxially electrospinning the collagen solution and at least one polymer to form collagen composite nanofibers, and collecting the collagen composite nanofibers. In some embodiments, for instance, the method may further comprise hydrating the collagen composite nanofibers and performing a cross-link reaction on the collagen composite nanofibers.

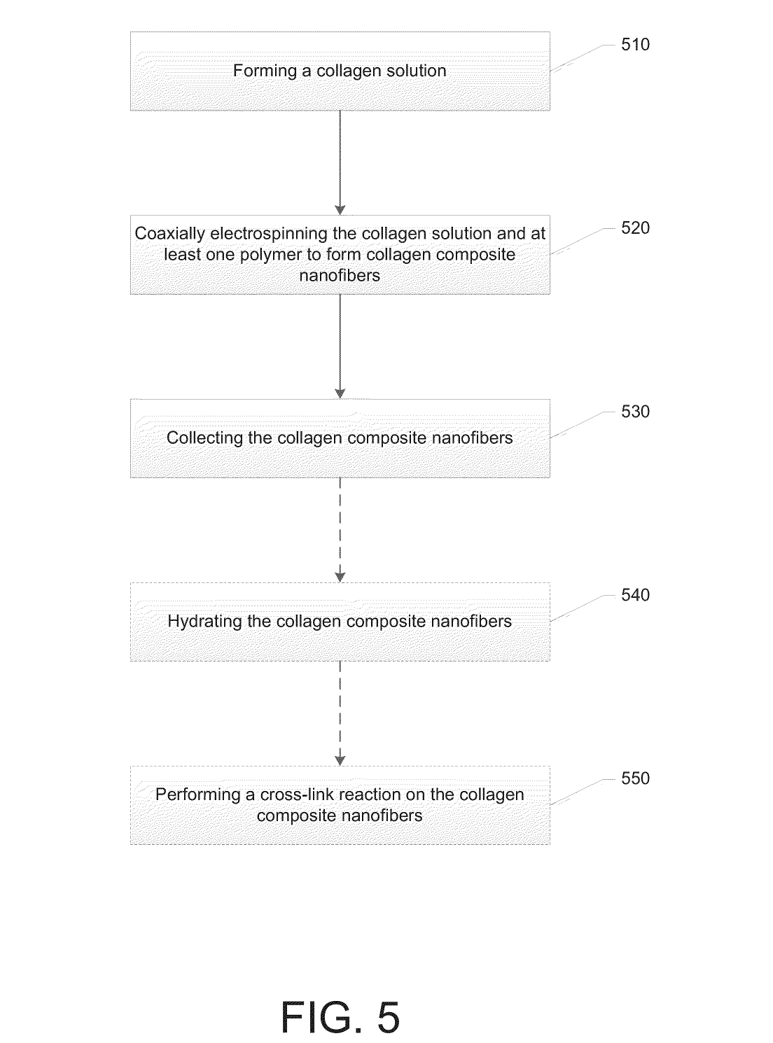

FIG. 5, for example, illustrates a block diagram of a method for preparing a collagen nanofibers including the optional steps of hydrating the collagen nanofibers and performing a cross-link reaction on the collagen nanofibers according to an example embodiment. As shown in FIG. 5, the exemplary method includes forming a collagen solution at operation 510, coaxially electrospinning the collagen solution and at least one polymer to form collagen nanofibers at operation 520, and collecting the collagen nanofibers at operation 530. The exemplary method may also include the optional steps of hydrating the collagen nanofibers at operation 540 and performing a cross-link reaction on the collagen composite nanofibers at operation 550.

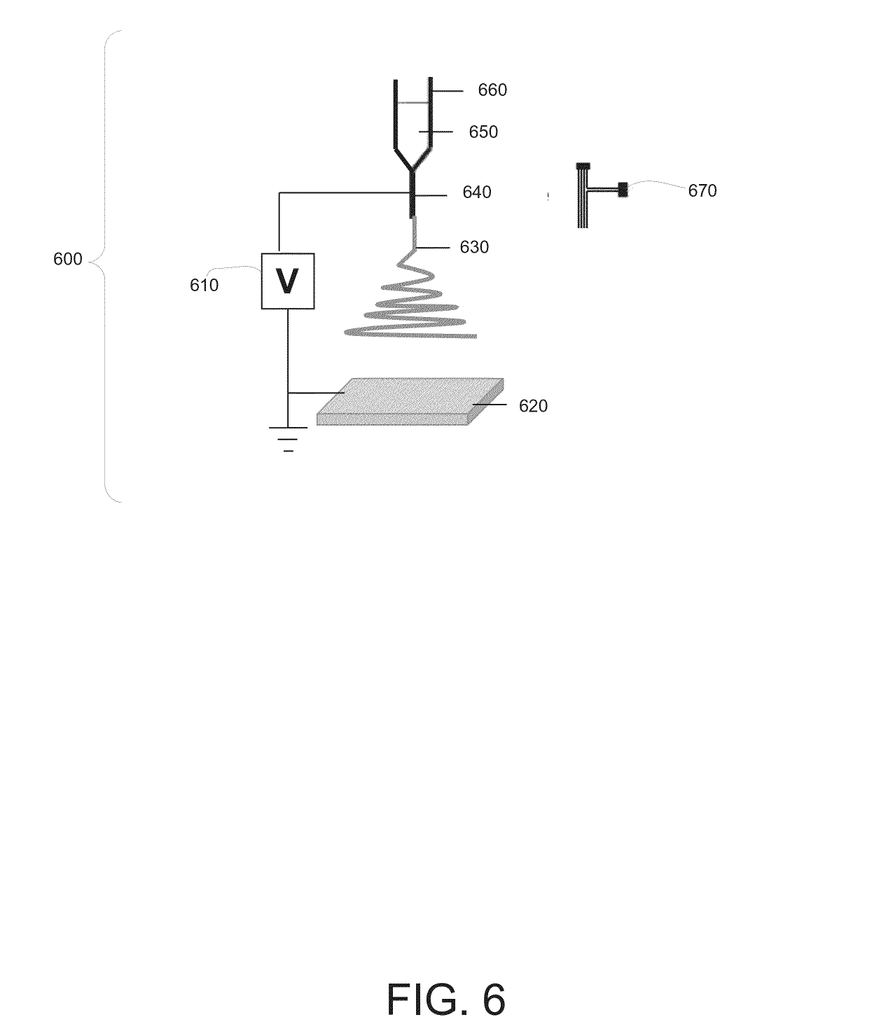

FIG. 6, for example, illustrates an electrospinning set-up according to an example embodiment. As shown in FIG. 6, the set-up 600 uses a high voltage power supply 610 to draw very fine collagen fibers (e.g., nanofibers) from a liquid collagen solution 650 stored in a syringe 660. In particular, when a sufficiently high voltage is applied to a liquid collagen solution 650 droplet in a needle 640, the body of the liquid collagen solution 650 becomes charged, and electrostatic repulsion counteracts the surface tension such that the droplet is stretched. In this regard, a charged liquid collagen solution jet 630 is formed. As the jet dries in flight, the mode of current flow changes from ohmic to convective as the charge migrates to the surface of the fiber. The jet is then elongated by a whipping process caused by electrostatic repulsion initiated at small bends in the fiber, until it is finally deposited on the grounded collector 620. The elongation and thinning of the fiber resulting from this bending instability leads to the formation of uniform collagen fibers with nanometer-scale diameters. In some embodiments, a coaxial needle 670 may be used to form, for example, a collagen composite.

According to certain embodiments, for example, forming the collagen solution may comprise dissolving collagen powder (e.g., Type I bovine collagen), in, by way of example only, 1,1,1,3,3,3-hexafluoro-2-propanol (HFP). In further embodiments, for instance, coaxially electrospinning the collagen solution and at least one polymer to form collagen composite nanofibers may comprise manipulating at least one of applied voltage, collector distance, collagen concentration, flow rate and/or the like to achieve the smallest collagen fibril diameter. In an example embodiment, for instance, the following conditions may be used: 7.75 wt. % collagen or 15.5 wt. % collagen, 0.5 mL/hr flow rate or 1 0 mL/hr flow rate, 15 kV applied voltage or 20 kV applied voltage, and 10 cm collector distance or 12 cm collector distance. As such, in certain embodiments, a fibril diameter from about 230 nm to about 370 nm may be achieved.

For example, according to certain embodiments, to achieve the smallest collagen fibril diameter, the collagen concentration may comprise from about 5 wt. % collagen to about 20 wt. % collagen. In other embodiments, for instance, the collagen concentration may comprise from about 6 wt. % collagen to about 15 wt. % collagen. In further embodiments, for example, the collagen concentration may comprise from about 7 wt. % collagen to about 10 wt. % collagen. In some embodiments, for instance, the collagen concentration may comprise about 8 wt. % collagen. As such, in certain embodiments, the collagen concentration may comprise from at least about any of the following: 5, 6, 7, and 8 wt. % collagen and/or at most about 20, 18, 15, 12, 10, 9, and 8 wt. % collagen (e.g., about 7-15 wt. % collagen, about 7-9 wt. % collagen, etc.).

In accordance with certain embodiments, for example, the flow rate may comprise from about 0.1 mL/hr to about 1.0 mL/hr. In other embodiments, for instance, the flow rate may comprise from about 0.2 mL/hr to about 0.8 mL/hr. In further embodiments, for example, the flow rate may comprise from about 0.4 mL/hr to about 0.6 mL/hr. In some embodiments, for instance, the flow rate may comprise of about 0.5 mL/hr. As such, in certain embodiments, the flow rate may comprise from at least about any of the following: 0.1, 0.2, 0.3, 0.4, and 0.5 mL/hr and/or at most about 1.0, 0.9, 0.8, 0.7, 0.6, and 0.5 mL/hr (e.g., about 0.3-0.9 mL/hr, about 0.4-0.8 mL/hr, etc.).

In accordance with certain embodiments, for example, the applied voltage may comprise from about 5 kV to about 30 kV. In other embodiments, for instance, the applied voltage may comprise from about 15 kV to about 25 kV. In further embodiments, for example, the applied voltage may comprise about 20 kV. As such, in certain embodiments, the applied voltage may comprise from at least about any of the following: 5, 8, 10, 12, 15, 18, and 20 kV and/or at most about 30, 28, 25, 22, and 20 kV (e.g., about 8-22 kV, about 20-25 kV, etc.).

In accordance with certain embodiments, for instance, the collector distance may comprise from about 5 cm to about 20 cm. In other embodiments, for example, the collector distance may comprise from about 8 cm to about 18 cm. In further embodiments, for instance, the collector distance may comprise from about 10 cm to about 15 cm. In certain embodiments, for example, the collector distance may comprise about 12 cm. As such, in certain embodiments, the collector distance may comprise from at least about any of the following: 5, 6, 7, 8, 9, 10, 11, and 12 cm and/or at most about 20, 19, 18, 17, 16, 15, 14, 13, and 12 cm (e.g., about 8-15 cm, about 10-20 cm, etc.).

According to certain embodiments, for example, the collagen composite nanofibers may comprise a fiber diameter from about 350 nm to about 950 nm. In other embodiments, for instance, the collagen composite nanofibers may comprise a fiber diameter from about 365 nm to about 945 nm. In further embodiments, for example, the collagen composite nanofibers may comprise a fiber diameter from about 380 nm to about 860 nm. In some embodiments, for instance, the collagen composite nanofibers may comprise a fiber diameter from about 500 nm to about 700 nm. In certain embodiments, for example, the collagen composite nanofibers may comprise a fiber diameter of about 620 nm. As such, in certain embodiments, the collagen composite nanofibers may comprise a fiber diameter from at least about any of the following: 350, 365, 380, 400, 450, 500, 550, 600, and 620 nm and/or at most about 950, 945, 925, 900, 860, 850, 800, 750, 700, 650, and 620 nm (e.g., about 400-700 nm, about 350-620 nm, etc.).

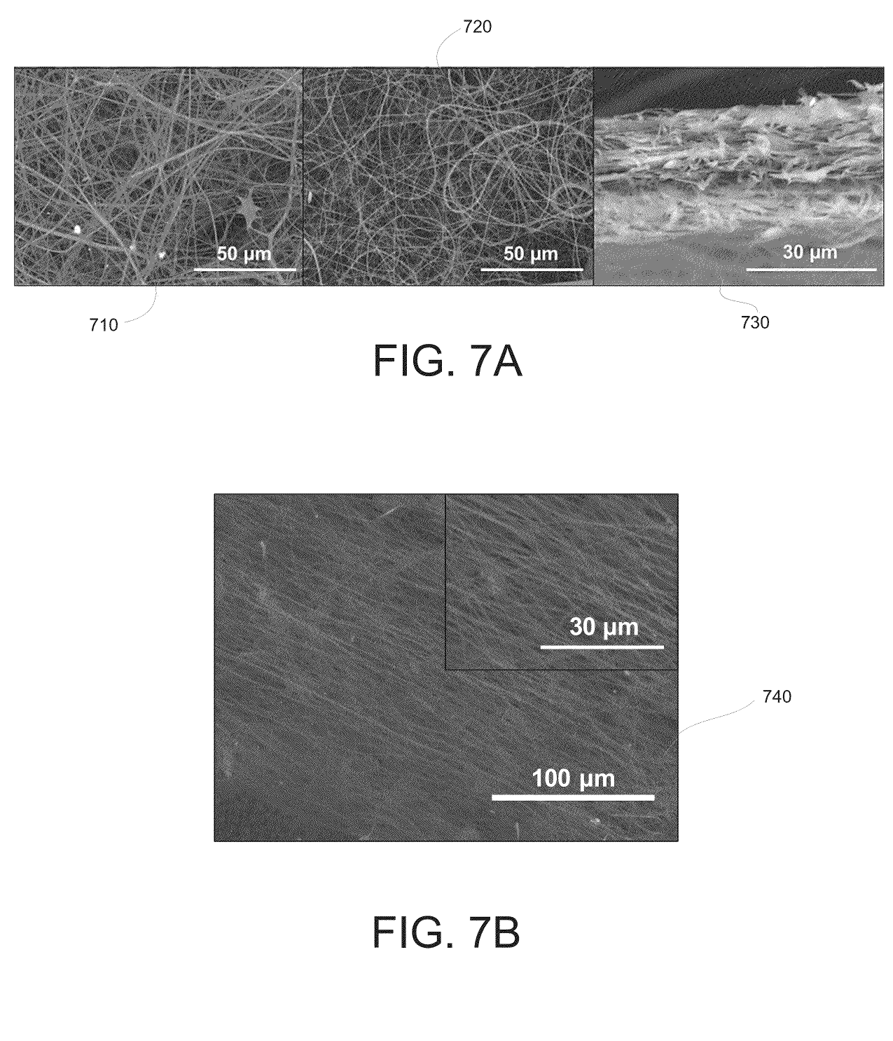

FIGS. 7A and 7B, for example, illustrate SEM images of electrospun collagen nanofibers according to an example embodiment. As shown in FIGS. 7A and 7B, by altering the electrospinning conditions such as applied voltage, collector distance, collagen concentration, flow rate and/or the like, different fibril diameters may be achieved. For example, in FIG. 7A SEM image 710 illustrates electrospun collagen nanofibers formed from 7.75 wt. % collagen, a flow rate of 1.0 mL/hr, an applied voltage of 15 kV, and a collector distance of 10 cm. In contrast, SEM image 720 illustrates electrospun collagen nanofibers formed from 7.75 wt. % collagen, a flow rate of 0 5 mL/hr, an applied voltage of 20 kV, and a collector distance of 12 cm. Accordingly, the formation conditions may be altered to form differently sized collagen nanofibers. SEM image 730 illustrates a cross section of electrospun collagen nanofibers to demonstrate their alignment. Moreover, SEM image 740 illustrates two different magnifications of electrospun collagen nanofibers that have been collected on a rotating drum, thereby further enhancing their alignment.

In further embodiments, for instance, the collagen composite nanofibers may be substantially aligned. In certain embodiments, for example, collecting the collagen composite nanofibers may comprise collecting the collagen composite nanofibers with a rotating drum as the collector. In this regard, for instance, the rotating drum may align the collagen composite nanofibers. As such, the collagen composite nanofibers may mimic the morphology of the cornea.

III. Collagen Composite

In yet another aspect, a collagen composite is provided. In general, the collagen composite, according to certain embodiments, may include a collagen shell and a polymer core. In such embodiments, for instance, the polymer core may comprise at least one therapeutic or sensory polymeric composition. Moreover, in such embodiments, for example, the collagen shell and the polymer core may be coaxially electrospun together to form the collagen composite nanofibers.

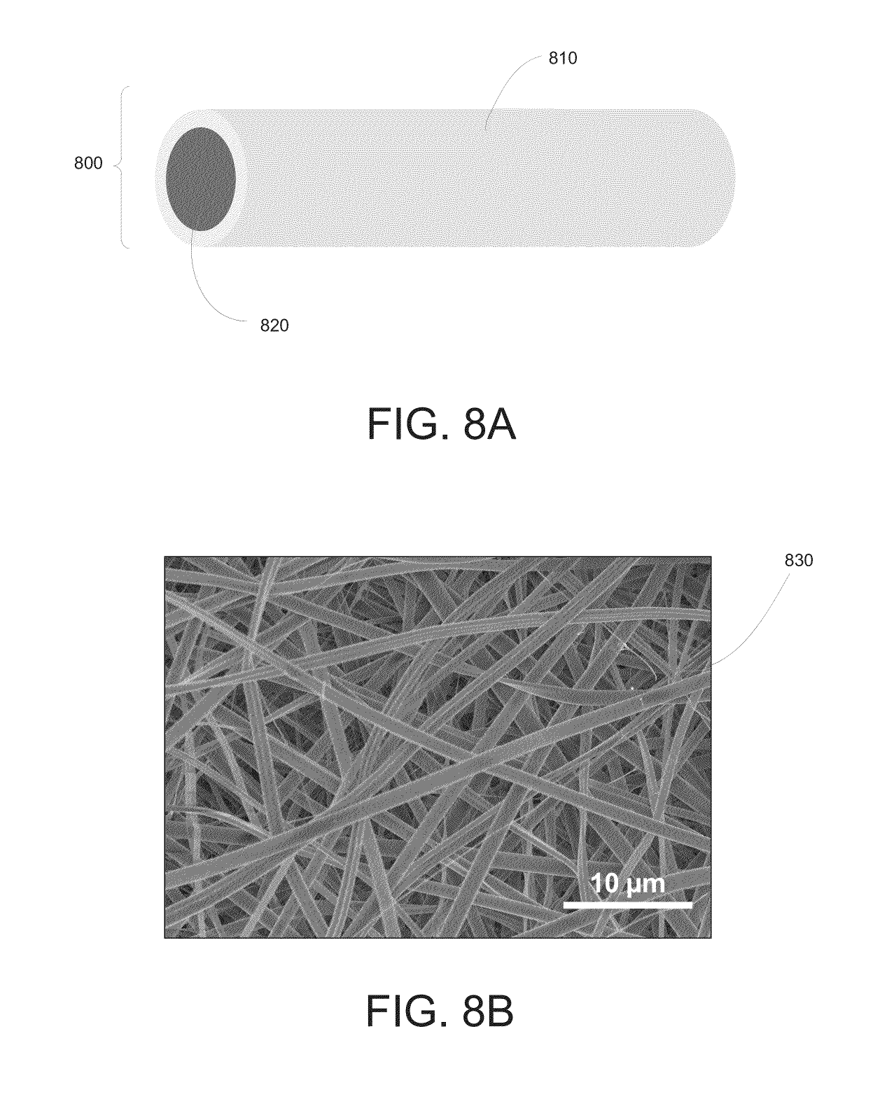

FIGS. 8A and 8B, for example, illustrate the structure and formation of collagen composite nanofibers according to an example embodiment. As shown in FIG. 8A, the material included in the polymer core may be combined with the material included in the collagen shell such that both materials are coaxially electrospun together in a manner that positions the entirety of the polymer core material within the collagen shell material. In this regard, a collagen composite 800 may be formed having a collagen shell 810 enveloping a polymer core 820. Similarly, FIG. 8B illustrates an SEM image 830 of collagen composite nanofibers.

In accordance with an example embodiment, for instance, the at least one therapeutic or sensory polymeric composition may comprise at least one of a collagen, a cellulose, a water-soluble polymer, or any combination thereof. In some embodiments, for example, the water-soluble polymer may comprise a drug-delivering polymer or sensory polymer, and the polymer core may further comprise at least one drug or sensory embedded in the drug-delivering polymer or sensory polymer.

In accordance with an example embodiment, for instance, the collagen composite nanofibers may be prepared by forming a collagen solution, coaxially electrospinning the collagen solution and at least one polymer to form collagen composite nanofibers, and collecting the collagen composite nanofibers. In some embodiments, for instance, the method may further comprise hydrating the collagen composite nanofibers and performing a cross-link reaction on the collagen composite nanofibers.

According to certain embodiments, for example, forming the collagen solution may comprise dissolving collagen powder (e.g., Type I bovine collagen), in, by way of example only, 1,1,1,3,3,3-hexafluoro-2-propanol (HFP). In further embodiments, for instance, coaxially electrospinning the collagen solution and at least one polymer to form collagen composite nanofibers may comprise manipulating at least one of applied voltage, collector distance, collagen concentration, flow rate and/or the like to achieve the smallest collagen fiber diameter. In an example embodiment, for instance, the following conditions may be used: 8 wt. % collagen, 0.5 mL/hr flow rate, 20 kV applied voltage, and 12 cm collector distance.

For example, according to certain embodiments, to achieve the smallest collagen fiber diameter, the collagen concentration may comprise from about 5 wt. % collagen to about 20 wt. % collagen. In other embodiments, for instance, the collagen concentration may comprise from about 6 wt. % collagen to about 15 wt. % collagen. In further embodiments, for example, the collagen concentration may comprise from about 7 wt. % collagen to about 10 wt. % collagen. In some embodiments, for instance, the collagen concentration may comprise about 8 wt. % collagen. As such, in certain embodiments, the collagen concentration may comprise from at least about any of the following: 5, 6, 7, and 8 wt. % collagen and/or at most about 20, 18, 15, 12, 10, 9, and 8 wt. % collagen (e.g., about 7-15 wt. % collagen, about 7-9 wt. % collagen, etc.).

In accordance with certain embodiments, for example, the flow rate may comprise from about 0.1 mL/hr to about 1.0 mL/hr. In other embodiments, for instance, the flow rate may comprise from about 0.2 mL/hr to about 0.8 mL/hr. In further embodiments, for example, the flow rate may comprise from about 0.4 mL/hr to about 0.6 mL/hr. In some embodiments, for instance, the flow rate may comprise of about 0.5 mL/hr. As such, in certain embodiments, the flow rate may comprise from at least about any of the following: 0.1, 0.2, 0.3, 0.4, and 0.5 mL/hr and/or at most about 1.0, 0.9, 0.8, 0.7, 0.6, and 0.5 mL/hr (e.g., about 0.3-0.9 mL/hr, about 0.4-0.8 mL/hr, etc.).

In accordance with certain embodiments, for example, the applied voltage may comprise from about 5 kV to about 30 kV. In other embodiments, for instance, the applied voltage may comprise from about 15 kV to about 25 kV. In further embodiments, for example, the applied voltage may comprise about 20 kV. As such, in certain embodiments, the applied voltage may comprise from at least about any of the following: 5, 8, 10, 12, 15, 18, and 20 kV and/or at most about 30, 28, 25, 22, and 20 kV (e.g., about 8-22 kV, about 20-25 kV, etc.).

In accordance with certain embodiments, for instance, the collector distance may comprise from about 5 cm to about 20 cm. In other embodiments, for example, the collector distance may comprise from about 8 cm to about 18 cm. In further embodiments, for instance, the collector distance may comprise from about 10 cm to about 15 cm. In certain embodiments, for example, the collector distance may comprise about 12 cm. As such, in certain embodiments, the collector distance may comprise from at least about any of the following: 5, 6, 7, 8, 9, 10, 11, and 12 cm and/or at most about 20, 19, 18, 17, 16, 15, 14, 13, and 12 cm (e.g., about 8-15 cm, about 10-20 cm, etc.).

According to certain embodiments, for instance, the collagen composite may comprise collagen composite nanofibers, the collagen composite nanofibers comprising a fiber diameter from about 350 nm to about 950 nm. In other embodiments, for instance, the collagen composite nanofibers may comprise a fiber diameter from about 365 nm to about 945 nm. In further embodiments, for example, the collagen composite nanofibers may comprise a fiber diameter from about 380 nm to about 860 nm. In some embodiments, for instance, the collagen composite nanofibers may comprise a fiber diameter from about 500 nm to about 700 nm. In certain embodiments, for example, the collagen composite nanofibers may comprise a fiber diameter of about 620 nm. As such, in certain embodiments, the collagen composite nanofibers may comprise a fiber diameter from at least about any of the following: 350, 365, 380, 400, 450, 500, 550, 600, and 620 nm and/or at most about 950, 945, 925, 900, 860, 850, 800, 750, 700, 650, and 620 nm (e.g., about 400-700 nm, about 350-620 nm, etc.).

In certain embodiments, for instance, the collagen composite nanofibers may be substantially aligned. In certain embodiments, for example, collecting the collagen composite nanofibers may comprise collecting the collagen composite nanofibers with a rotating drum as the collector. In this regard, for instance, the rotating drum may align the collagen composite nanofibers. As such, the collagen composite nanofibers may mimic the morphology of the cornea.

Exemplary Embodiments

Having described various aspects and exemplary embodiments herein, further specific embodiments include those set forth in the following paragraphs.

In some example embodiments, a method for preparing a collagen membrane is provided. In general, the method for preparing the collagen membrane, according to certain embodiments, comprises applying an influence of an electric field to an acidic collagen solution positioned between two capacitor plates, adding a buffer solution to the acidic collagen solution to form a collagen gel, assembling a plurality of collagen gel layers, and performing a dehydrothermal cross-link on the collagen gel to form a cross-linked collagen membrane. In certain embodiments, the method further comprises rehydrating the cross-linked collagen membrane. In some embodiments, the method further comprises performing a chemical cross-linking reaction on the cross-linked collagen membrane to form a double cross-linked collagen membrane and rehydrating the double cross-linked collagen membrane.

In accordance with an example embodiment, applying the influence of the electric field to the acidic collagen solution positioned between the capacitor plates comprises applying a voltage between parallel capacitor plates for a time period of about 1 hour to about 15 hours. In some embodiments, applying the influence of the electric field to the acidic collagen solution positioned between the capacitor plates comprises applying a voltage at an electrode distance from about 0.1 cm and about 5 cm. In certain embodiments, applying the influence of the electric field to the acidic collagen solution positioned between the capacitor plates comprises applying a voltage from about 1 V to about 15 V.

In accordance with an example embodiment, the collagen membrane is substantially transparent. In some embodiments, the collagen membrane comprises a plurality of collagen fibrils, and the plurality of collagen fibrils are substantially aligned. In further embodiments, the collagen membrane comprises a thickness from about 100 microns to about 600 microns. As such, in certain embodiments, the thickness is substantially the same as a corneal thickness. Accordingly, in some embodiments, the collagen membrane is configured to be sutured to an eye.

In another aspect, a method for preparing a collagen composite is provided. In general, the method for preparing a collagen composite, according to certain embodiments, comprises forming a collagen solution, coaxially electrospinning the collagen solution and at least one polymer to form collagen composite nanofibers, and collecting the collagen composite nanofibers. In some embodiments, the method further comprises hydrating the collagen composite nanofibers and performing a cross-link reaction on the collagen composite nanofibers. In certain embodiments, the collagen composite nanofibers comprise a fiber diameter from about 350 nm to about 950 nm. In further embodiments, the collagen composite nanofibers are substantially aligned.

In yet another aspect, a collagen composite is provided. In general, the collagen composite, according to certain embodiments, comprises a collagen shell and a polymer core. In such embodiments, the polymer core comprises at least one therapeutic or sensory polymeric composition. Moreover, in such embodiments, the collagen shell and the polymer core are coaxially electrospun together to form the collagen composite.

In accordance with an example embodiment, the at least one therapeutic or sensory polymeric composition comprises at least one of a collagen, a cellulose, a water-soluble polymer, or any combination thereof. In some embodiments, the water-soluble polymer comprises a drug-delivering polymer or sensory polymer, and the polymer core further comprises at least one drug or sensory embedded in the drug-delivering polymer or sensory polymer. In further embodiments, the collagen composite comprises collagen composite nanofibers, the collagen composite nanofibers comprising a fiber diameter from about 350 nm to about 950 nm. In certain embodiments, the collagen composite nanofibers are substantially aligned.

Many modifications and other embodiments will come to mind to one skilled in the art to which this disclosure pertains having the benefit of the teachings presented in the foregoing descriptions and the associated drawings. Therefore, it is to be understood that this disclosure is not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

That which is claimed:

1. A method for preparing a collagen membrane, the method comprising:
applying an influence of an electric field to an acidic collagen solution positioned between two capacitor plates;
adding a buffer solution to the acidic collagen solution to form a collagen gel;
assembling a plurality of collagen gel layers; and
performing a dehydrothermal cross-link on the plurality of collagen gel layers to form a cross-linked collagen membrane.

2. The method according to claim 1, further comprising rehydrating the cross-linked collagen membrane.

3. The method according to claim 2, further comprising:
performing a chemical cross-linking reaction on the cross-linked collagen membrane to form a double cross-linked collagen membrane; and
rehydrating the double cross-linked collagen membrane.

4. The method according to claim 1, wherein applying the influence of the electric field to the acidic collagen solution positioned between the capacitor plates comprises applying a voltage between the capacitor plates for a time period of about 1 hour to about 15 hours.

5. The method according to claim 1, wherein applying the influence of the electric field to the acidic collagen solution positioned between the capacitor plates comprises applying a voltage at an electrode distance from about 0.1 cm and about 5 cm.

6. The method according to claim 1, wherein applying the influence of the electric field to the acidic collagen solution positioned between the capacitor plates comprises applying a voltage from about 1 V to about 15 V.

7. The method according to claim 1, wherein the collagen membrane is substantially transparent.

8. The method according to claim 1, wherein the collagen membrane comprises a plurality of collagen fibrils, and the plurality of collagen fibrils are substantially aligned.

9. The method according to claim 1, wherein the collagen membrane comprises a thickness from about 100 microns to about 600 microns.

10. The method according to claim 9, wherein the thickness is substantially the same as a corneal thickness.

11. The method according to claim 1, wherein the collagen membrane is configured to be sutured to an eye.

12. A method for preparing a collagen composite, the method comprising:
forming a collagen solution;
coaxially electrospinning the collagen solution and at least one polymer to form collagen composite nanofibers; and
collecting the collagen composite nanofibers.

13. The method according to claim 12, further comprising:
hydrating the collagen composite nanofibers; and
performing a cross-link reaction on the collagen composite nanofibers.

14. The method according to claim 12, wherein the collagen composite nanofibers comprise a fiber diameter from about 350 nm to about 950 nm.

15. The method according to claim 12, wherein the collagen composite nanofibers are substantially aligned.

16. A collagen composite, comprising:
a collagen shell; and
a polymer core, the polymer core comprising at least one of a therapeutic polymeric composition, a sensory polymeric composition, or any combination thereof,
wherein the collagen shell and the polymer core are coaxially electrospun together to form the collagen composite.

17. The collagen composite according to claim 16, wherein the at least one of the therapeutic polymeric composition, the sensory polymeric composition, or any combination thereof comprises at least one of a collagen, a cellulose, a water-soluble polymer, or any combination thereof.

18. The collagen composite according to claim 17, wherein the water-soluble polymer comprises a drug-delivering polymer or sensory polymer, and the polymer core further comprises at least one drug or sensory embedded in the drug-delivering polymer or sensory polymer.

19. The collagen composite according to claim 16, wherein the collagen composite comprises collagen composite nanofibers, the collagen composite nanofibers comprising a fiber diameter from about 350 nm to about 990 nm.

20. The collagen composite according to claim 19, wherein the collagen composite nanofibers are substantially aligned.
